(19) United States
(12) Patent Application Publication
(10) Pub. No.: US 2021/0121542 A1
(43) Pub. Date: Apr. 29, 2021

(54) COMPOSITION FOR DELIVERY OF PROTEIN THERAPEUTICS THROUGH ORAL, SUBLINGUAL AND BUCCAL ROUTE

(71) Applicant: Prime Bio, Inc., Dartmouth, MA (US)

(72) Inventors: Bal Ram Singh, Dartmouth, MA (US); Raj Kumar, Dartmouth, MA (US); Kruti Patel, Dartmouth, MA (US)

(21) Appl. No.: 17/082,035

(22) Filed: Oct. 28, 2020

Related U.S. Application Data
(60) Provisional application No. 62/926,640, filed on Oct. 28, 2019.

(51) Int. Cl. A61K 38/48

(52) U.S. Cl. CPC A61K 38/4893; C12Y 304/24069

(57) ABSTRACT

The present invention relates to a composition for the delivery of drug molecules, especially therapeutic proteins via oral route, sublingual, and buccal routes. The embodiments herein provide delivery of therapeutic proteins with the help of neurotoxin associated proteins (NAPs) from Clostridium botulinum type A or type E. The NAPs combine with proteins and/or drug molecules to form a composition for oral or sublingual delivery.

### BACKGROUND

### Technical Field of Invention

The embodiments herein generally relate to protein delivery system and particularly to the delivery of protein therapeutics through oral, sublingual and buccal route. The embodiments herein more particularly relate to a system and composition comprising Neurotoxin Associated Proteins (NAP's) for a successful delivery of protein therapeutics.

### DESCRIPTION OF RELATED ART

Therapeutic proteins have been widely used for treating different conditions. The therapeutic proteins are usually grouped based on their molecular types which include antibody-based drugs, anticoagulants, blood factors, bone morphogenetic proteins, engineered protein scaffolds, enzymes, Fc fusion proteins, growth factors, hormones, interferons, interleukins, and thrombolytics.

The major route of therapeutic proteins administration is through parenteral, mainly through IV and IM and subcutaneous injections. However, these can lead to infection at the site of injection. In addition, injection needs to be administered through health care professionals. Thus, it is preferred to administer the proteins through the oral or sublingual routes. The therapeutic proteins are delicate molecules with high 3D structures held through non-covalent bonds. The 3D structures are very critical for maintaining the protein's biological functions.

Once the protein therapeutics reaches the GI tract environment, it has to come across harsh condition such as high acidic pH. Further, the proteins get degraded by proteases.

Hence there is a need to develop a delivery system for protein therapeutics from oral, sublingual or buccal route so that the problem of harsh digestive conditions in stomach like very low pH and bile acid, and proteases can be solved, and the proteins get delivered to the body easily and successfully without getting disrupted.

Botulinum neurotoxins (BoNT) are complexes which are produced by Gram-positive, anaerobic, and soporiferous Clostridium botulinum. BoNT are capable of entering the body through respiratory tract, gastrointestinal tract, while wounds form the main entrances for BoNT. Based on the antigenic properties, target sites, differences in structures, and toxicity, these neurotoxins are classified into seven serotypes: A, B, C, D, E, F, and G. Serotypes A, B, E, and rarely F cause illness in humans, whereas serotypes C and D cause illness in animals.

The Neurotoxin Associated Proteins (NAP's) such as Botulinum neurotoxins (BoNT) can be combined with the protein therapeutics for their safe delivery in the blood through oral, sublingual or buccal route.

In view of foregoing, there is a need to come up with a composition or a method of synthesizing a safer delivery system for the protein therapeutics using Neurotoxin Associated Proteins (NAPs) from Clostridium botulinum as adjuvants.

The above-mentioned shortcomings, disadvantages and problems are addressed herein, as detailed below.

### SUMMARY OF THE INVENTION

Thus, the primary object of the embodiments herein is to provide a composition comprising protein therapeutics and a Neurotoxin Associated Protein (NAP) which can be taken orally, sublingually or through buccal route.

Yet another object of the embodiments herein is to provide a composition for oral or sublingual administrations of protein therapeutics which can withstand the harsh conditions of the GI tract and can translocate through the epithelial cells of the GI tract.

Yet another object of the embodiments herein is to provide system or a composition that solves the problem of safety concerns related to the injection of vaccines by not requiring an injection.

According to an embodiment herein, a composition for delivery of protein therapeutics through oral, sublingual and buccal routes comprises at least one protein therapeutics and a Neurotoxin Associated Protein (NAPs).

According to an embodiment herein, the protein therapeutics comprises one or more protein selected from the group consisting of peptide/protein hormones, vaccines, therapeutics enzymes, monoclonal antibodies, cytokines, blood factors, and/or peptide antibiotics.

According to an embodiment herein, the NAPs is capable of binding to the protein therapeutics.

According to an embodiment herein, the NAPs is derived from botulinum neurotoxin.

According to an embodiment herein, the botulinum neurotoxin is derived from botulinum neurotoxin A and botulinum neurotoxin E.

According to an embodiment herein, the NAP is selected from the group consisting of Hn-33 of botulinum neurotoxin A or P-80 of botulinum neurotoxin E.

According to an embodiment herein, the Hn-33 of botulinum neurotoxin is a recombinant Hn-33 (rHn-33).

According to an embodiment herein, the P-80 of botulinum neurotoxin is recombinant P-80 (rP-80).

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows the transport of insulin through the CaCO-2 cell monolayer, according to an embodiment herein.

FIG. 2 shows BSA protection against trypsin using Hn-33 and P80 at 2 hours, according to an embodiment herein.

### DETAILED DESCRIPTION OF THE DRAWINGS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The embodiments herein provide a composition for the delivery of drug molecules, especially therapeutic proteins via oral route, sublingual, and buccal routes. The embodiments herein provide delivery of therapeutic proteins with the help of neurotoxin associated proteins (NAPs) from Clostridium botulinum type A or type E. The NAPs combine with proteins and/or drug molecules to form a composition for oral or sublingual delivery.

According to an embodiment herein, a composition for delivery of protein therapeutics through oral, sublingual and buccal routes comprises at least one protein therapeutics and a Neurotoxin Associated Protein (NAPs).

According to an embodiment herein, the protein therapeutics comprises one or more protein selected from the group consisting of peptide/protein hormones, vaccines, therapeutics enzymes, monoclonal antibodies, cytokines, blood factors, and/or peptide antibiotics.

According to an embodiment herein, the NAPs is capable of binding to the protein therapeutics.

According to an embodiment herein, the neurotoxin associated protein is derived from botulinum neurotoxin.

According to an embodiment herein, the botulinum neurotoxin is derived from botulinum neurotoxin A and botulinum neurotoxin E.

According to an embodiment herein, the neurotoxin associated protein is selected from the group consisting of Hn-33 of botulinum neurotoxin A or P-80 of botulinum neurotoxin E.

According to an embodiment herein, the Hn-33 of botulinum neurotoxin is a recombinant Hn-33 (rHn-33).

According to an embodiment herein, the P-80 of botulinum neurotoxin is recombinant P-80 (rP-80).

According to an embodiment herein, the protein is selected from detoxified recombinant botulinum neurotoxin or insulin.

According to another embodiment herein, a composition for delivery of protein therapeutics through oral, sublingual and buccal routes comprises neurotoxin associated protein and a detoxified recombinant botulinum neurotoxin.

According to another embodiment herein, a composition for delivery of protein therapeutics through oral, sublingual and buccal routes comprises a neurotoxin associated protein and an insulin.

According to another embodiment herein, a method of treatment of a disease or a condition related to a protein therapeutic by administering a composition comprising the protein therapeutic and a Neurotoxin Associated Protein.

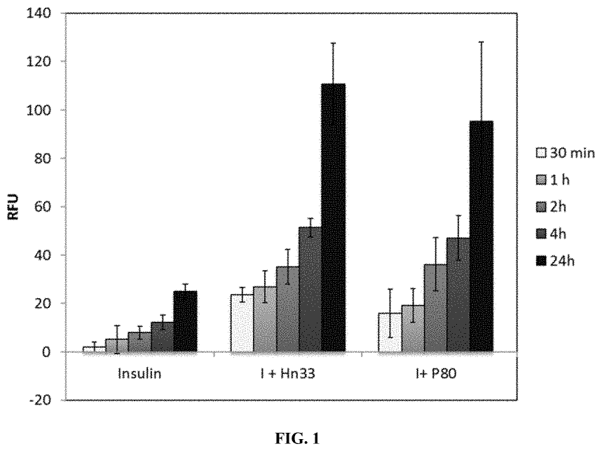

FIG. 1 shows the transport of insulin through the CaCO-2 cell monolayer, according to an embodiment herein. With respect to FIG. 1, it is evident that the transport of the insulin has enhanced multiple fold with the help of Hn33 and P80. The NAPs protect the insulin from degradation and helps in translocation across the CaCo-2 cell monolayer.

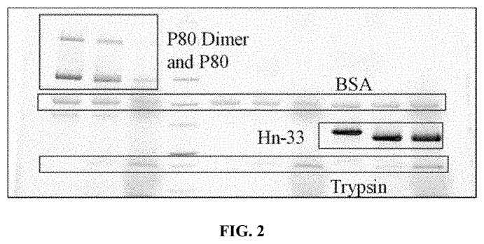

FIG. 2 shows BSA protection against trypsin using Hn-33 and P80 at 2 hours, according to an embodiment herein. With respect to FIG. 2, the Lane 1 is for 0.2 mg/mL P80-BSA control, Lane 2 is for 0.1 mg/mL trypsin+0.2 mg/mL P80-BSA, Lane 3 is for 0.5 mg/mL trypsin+0.2 mg/mL P80-BSA. Lane 4 is for MW marker, Lane 5 is for 0.2 mg/mL BSA control, Lane 6 is for 0.1 mg/mL trypsin+0.2 mg/mL BSA, Lane 7 is for 0.5 mg/mL trypsin+0.2 mg/mL BSA, Lane 8 is for Hn33-BSA control, Lane 9 is for 0.1 mg/mL trypsin+0.2 mg/mL Hn33-BSA, and Lane 10: 0.5 mg/mL trypsin+0.2 mg/mL Hn33-BSA.

TABLE 1
BSA protection against trypsin using Hn-33 and P80 at 2 hours

| Experiment | Intensity Relative | % Relative Protection |
|---|---|---|
| Conditions | to Control (%) | (compared with BSA + Trypsin) |
| BSA 0.1 mg/mL | 75 | 0 |
| Trypsin | | |
| BSA 0.5 mg/mL | 69 | 0 |
| Trypsin | | |
| BSA-P80 | 86 | 13 |
| 0.1 mg/mL Trypsin | | |
| BSA-P80 | 64 | −6 |
| 0.5 mg/mL Trypsin | | |
| BSA-Hn33 | 89 | 16 |
| 0.1 mg/mL Trypsin | | |
| BSA-Hn33 | 90 | 23 |
| 0.5 mg/mL Trypsin | | |

FIG. 2 and table 1 discloses that the Hn-33 and P-80 protect bovine serum albumin (BSA) against proteases.

The presently invention employs the use of neurotoxin associated proteins (NAPs) of botulinum neurotoxin (BoNT) produced by Clostridium botulinum as delivery vehicles for the proteins. BoNT is a food poison produced in the form of a complex with NAPs, which protect it from the low pH and proteases of the gastro-intestinal tract. In addition, NAPs are known to help translocate the BoNT across the mucosal layer of intestine as well as the nasal passage. The NAPs can bind and protect and translocate a protein therapeutics across the mucosal layer.

Clostridium botulinum produces seven serotypes of botulinum neurotoxins (A-G). It is basically made of two protein chains one is 100 kDa polypeptide heavy chain and another is 50 kDa polypeptide light chain bridged through disulphide link. Botulinum neurotoxins is synthesized in a complex form, in which neurotoxin is surrounded by several non-toxin proteins known as neurotoxin associated proteins (NAPs). NAPs have been shown to have two major roles in the intoxication process of botulism. The first role is the assistance of NAPs in the translocation of the BoNT across the intestinal mucosal layer. The second role is NAPs protect the BoNT against acidity and proteolytic attack of the enzymes of gastric juice. Therefore, NAPs become a perfect delivery system for protein therapeutics.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims.

I claim:

1. A composition for delivery of protein therapeutics through oral, sublingual and buccal routes comprises:
at least one protein therapeutics; and
a Neurotoxin Associated Protein (NAPs).

2. The composition according to claim 1, wherein the protein therapeutics comprises one or more protein selected from the group consisting of peptide/protein hormones, vaccines, therapeutics enzymes, monoclonal antibodies, cytokines, blood factors, and/or peptide antibiotics.

3. The composition according to claim 1, wherein the NAPs is capable of binding to the protein therapeutics.

4. The composition according to claim 1, wherein the NAPs is derived from botulinum neurotoxin.

5. The composition according to claim 4, wherein the botulinum neurotoxin is derived from botulinum neurotoxin A and botulinum neurotoxin E.

6. The composition according to claims 1 and 4, wherein the NAP is selected from the group consisting of Hn-33 of botulinum neurotoxin A or P-80 of botulinum neurotoxin E.

7. The composition according to claim 6, wherein the Hn-33 of botulinum neurotoxin is a recombinant Hn-33 (rHn-33).

8. The composition according to claim 6, wherein the P-80 of botulinum neurotoxin is recombinant P-80 (rP-80).
